(19) United States
(12) Patent Application Publication
(10) Pub. No.: US 2014/0356802 A1
(43) Pub. Date: Dec. 4, 2014

(54) Intraoral Light Emitting Appliance

(71) Applicant: Lumed Science, Inc., Denver, CO (US)

(72) Inventors: Juraj Balog, Denver, CO (US); Michael S. Bojanowski, Denver, CO (US)

(21) Appl. No.: 13/907,744

(22) Filed: May 31, 2013

(51) Int. Cl. A61B 1/06; A61B 1/24

(52) U.S. Cl. CPC A61B 1/06; A61B 1/24
USPC 433/29

(57) ABSTRACT

An intraoral light emitting appliance having an external surface including opposed bite surfaces adapted for removable engagement by the upper and lower teeth to prop open the mouth of a patient.

### I. FIELD OF THE INVENTION

An intraoral light emitting appliance having an external surface including opposed bite surfaces adapted for removable engagement by the upper and lower teeth to prop open the mouth of a patient.

### II. BACKGROUND OF THE INVENTION

Conventionally, mouth props are inserted into the patient's mouth between upper and lower teeth to keep the mouth opened in a fixed position during performance of surgical, dental or other procedures in the patient's mouth. Illumination of the oral cavity may be provided by a light source located outside of the oral cavity with light directed toward the open mouth of the patient. Illumination of the oral cavity by an external light source can be disadvantaged by shadows resulting from hands and equipment interposed between the light source and the oral cavity of the patient.

Conventional illumination of the oral cavity by an intraoral light source may be disadvantaged by having a power source located outside of the oral cavity which requires an electrical power conduit between the power source and the intraoral light source which occupies a part of the mouth opening interfering with placement of hands and equipment and treatment of the patient.

Similarly, illumination of the oral cavity by use of a tube or a pipe which transmits light from a light source external to the oral cavity to a tube or pipe end located within the oral cavity requires that the tube or pipe occupy a part of the mouth interfering with placement of hands and equipment and treatment of the patient.

Additionally, conventional mouth props may not be compatible with procedures using temperatures and pressures, or using one or more antimicrobial fluids, to destroy microorganisms and are used once and discarded.

### III. SUMMARY OF THE INVENTION

Accordingly, a broad object of the invention is to provide an intraoral illumination appliance comprising an appliance body including an external surface having opposed bite surfaces adapted to be engaged by upper and lower teeth to prop open a mouth of a patient; an internal surface defining an interior chamber; and a translucent portion which communicates between the internal surface and the external surface of said appliance body; at least one light emitting element; and a power source electrically connected to said light emitting element, the light emitting element and the power source disposed entirely inside the interior chamber of the appliance body, the light emitting element having a location inside the interior chamber to emit light through the translucent portion of the appliance body.

Another broad object of the invention can be to provide a method of producing an intraoral illumination appliance comprising providing an appliance body including an external surface including opposed bite surfaces adapted to be engaged by the upper and lower teeth to prop open a mouth of a patient; an internal surface defining an interior chamber; and a translucent portion which communicates between the internal surface and the external surface of said appliance body; and disposing entirely within the interior chamber of the appliance body at least one light emitting element located to emit light through the translucent portion of the appliance body and a power source electrically connected to the light emitting element.

Another broad object of the invention can be to provide a method of using an intraoral illumination appliance comprising inserting the appliance into a mouth of a patient, the appliance including an external surface including opposed bite surfaces; an internal surface defining an interior chamber; and at least one translucent portion which communicates between the internal surface and the external surface of the appliance body; at least one light emitting element; and a power source electrically connected to the light emitting element, the light emitting element and the power source disposed entirely inside the interior chamber of the appliance body; engaging upper and lower teeth of the patient with the opposed bite surfaces of the appliance body to prop open the mouth; and emitting light through the at least one translucent portion of the appliance to illuminate the mouth of said patient by operation of the light emitting element.

### IV. BRIEF DESCRIPTION OF THE DRAWINGS

FIG. 1 is an illustration which shows a particular method of using a particular embodiment of the inventive intraoral illumination appliance.

FIG. 2 is a perspective first side view of a particular embodiment of the inventive intraoral illumination appliance.

FIG. 3 is perspective second side view of a particular embodiment of the inventive intraoral illumination appliance.

FIG. 4 is a perspective first side exploded view of the particular embodiment of the inventive intraoral illumination appliance shown in FIGS. 2 and 3.

FIG. 5 is a perspective second side exploded view of the particular embodiment of the inventive intraoral illumination appliance shown in FIGS. 2 and 3.

FIG. 6 is a first side view of the particular embodiment of the inventive intraoral illumination appliance shown in FIGS. 2 and 3.

FIG. 7 is a second side view of the particular embodiment of the inventive intraoral illumination appliance shown in FIGS. 2 and 3.

FIG. 8 is back view of the particular embodiment of the inventive intraoral illumination appliance shown in FIGS. 2 and 3.

FIG. 9 is front view of the particular embodiment of the inventive intraoral illumination appliance shown in FIGS. 2 and 3.

FIG. 10 is top view of the particular embodiment of the inventive intraoral illumination appliance shown in FIGS. 2 and 3.

FIG. 11 is a bottom view of the particular embodiment of the inventive intraoral illumination appliance shown in FIGS. 2 and 3.

FIG. 12 is a perspective first side view of another particular embodiment of the inventive intraoral illumination appliance.

FIG. 13 is a perspective second side view of another particular embodiment of the inventive intraoral illumination appliance.

FIG. 14 is a perspective first side exploded view of the particular embodiment of the inventive intraoral illumination appliance shown in FIGS. 12 and 13.

FIG. 15 is a perspective second side exploded view of the particular embodiment of the inventive intraoral illumination appliance shown in FIGS. 12 and 13.

FIG. 16 is a first side view of the particular embodiment of the inventive intraoral illumination appliance shown in FIGS. 12 and 13.

FIG. 17 is a second side view of the particular embodiment of the inventive intraoral illumination appliance shown in FIGS. 12 and 13.

FIG. 18 is a front view of the particular embodiment of the inventive intraoral illumination appliance shown in FIGS. 12 and 13.

FIG. 19 is a back view of the particular embodiment of the inventive intraoral illumination appliance shown in FIGS. 12 and 13.

FIG. 20 is a top view of the particular embodiment of the inventive intraoral illumination appliance shown in FIGS. 12 and 13.

FIG. 21 is a bottom view of the particular embodiment of the inventive intraoral illumination appliance shown in FIGS. 12 and 13.

FIG. 22 is first side perspective view which illustrates the particular embodiment shown in FIGS. 12 and 13 having a retention element adapted to retain one or more discrete intraoral devices.

FIG. 23 is second side perspective view which illustrates the particular embodiment shown in FIGS. 12 and 13 having a retention element adapted to retain one or more discrete intraoral devices.

FIG. 24 is back side view which illustrates the particular embodiment shown in FIGS. 12 and 13 having a retention element adapted to retain one or more discrete intraoral devices.

FIG. 25 is top view which illustrates the particular embodiment shown in FIGS. 12 and 13 having a retention element adapted to retain one or more discrete intraoral devices.

### V. DETAILED DESCRIPTION OF THE INVENTION

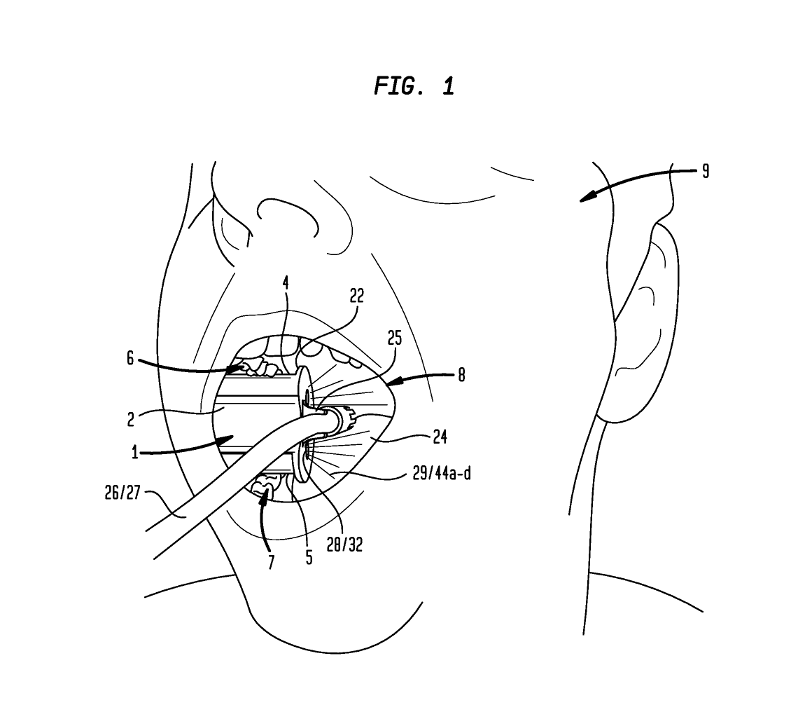

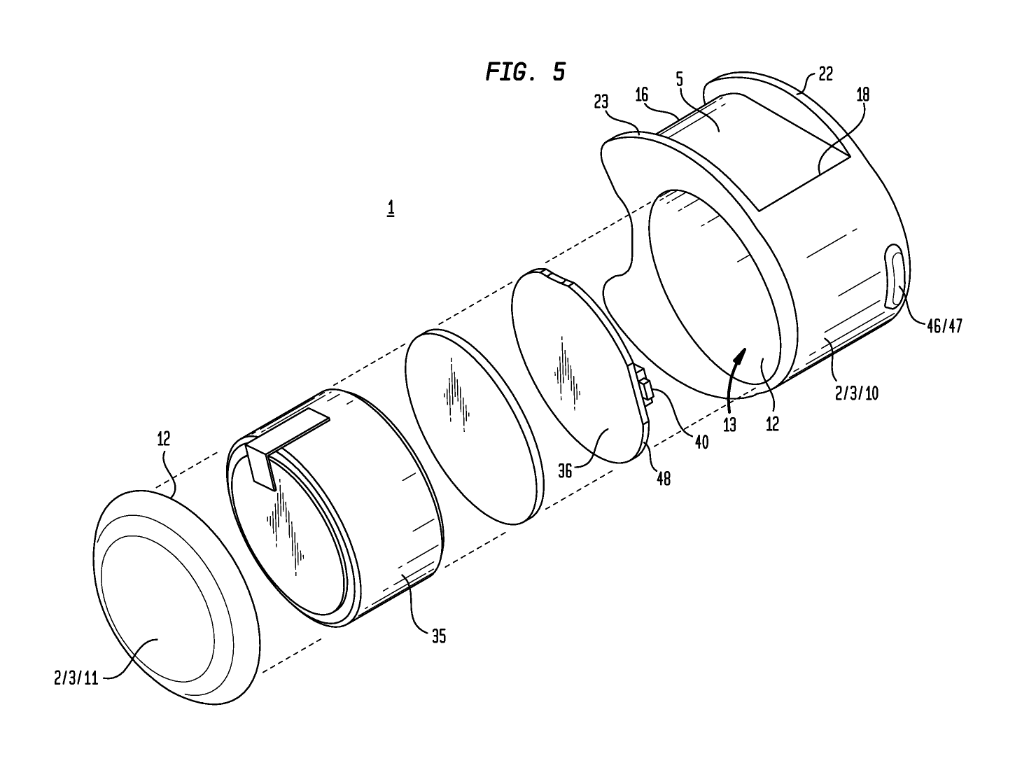

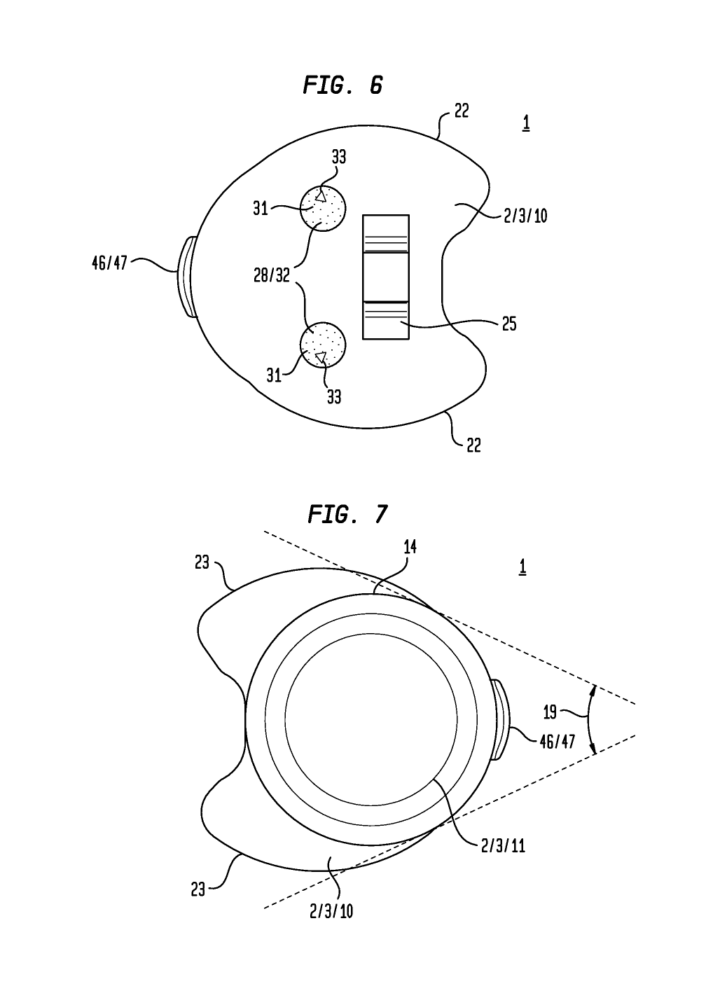

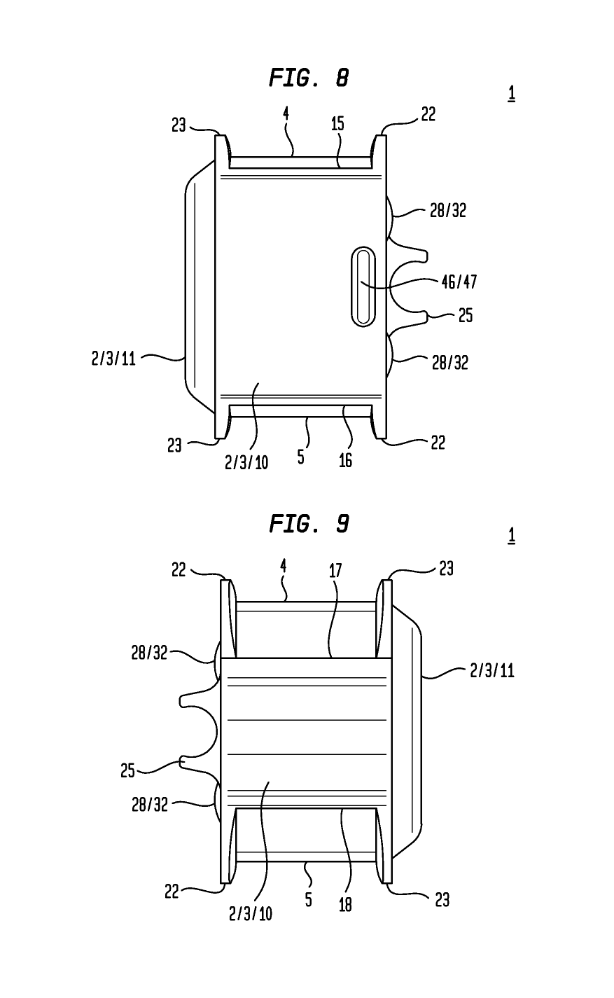

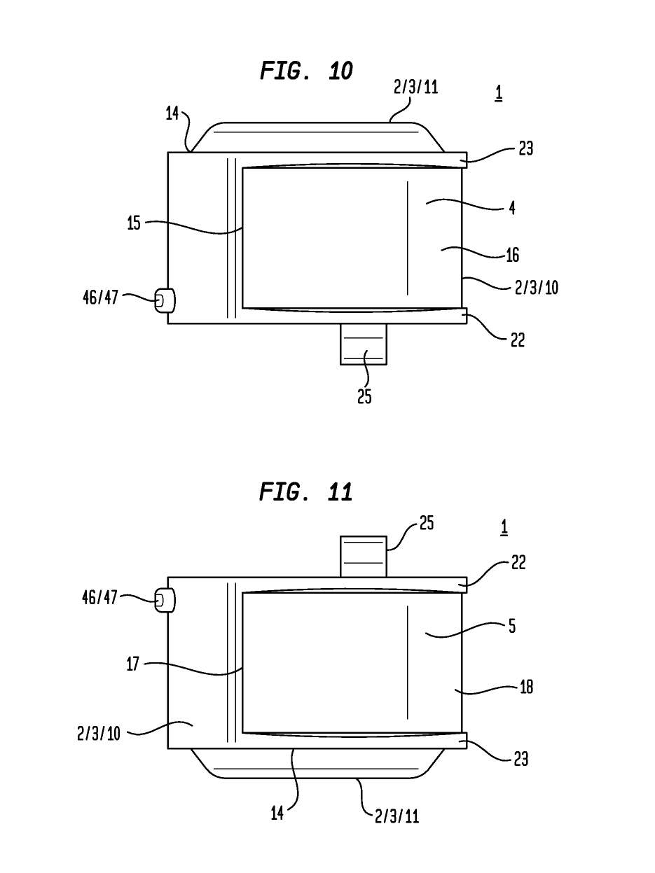

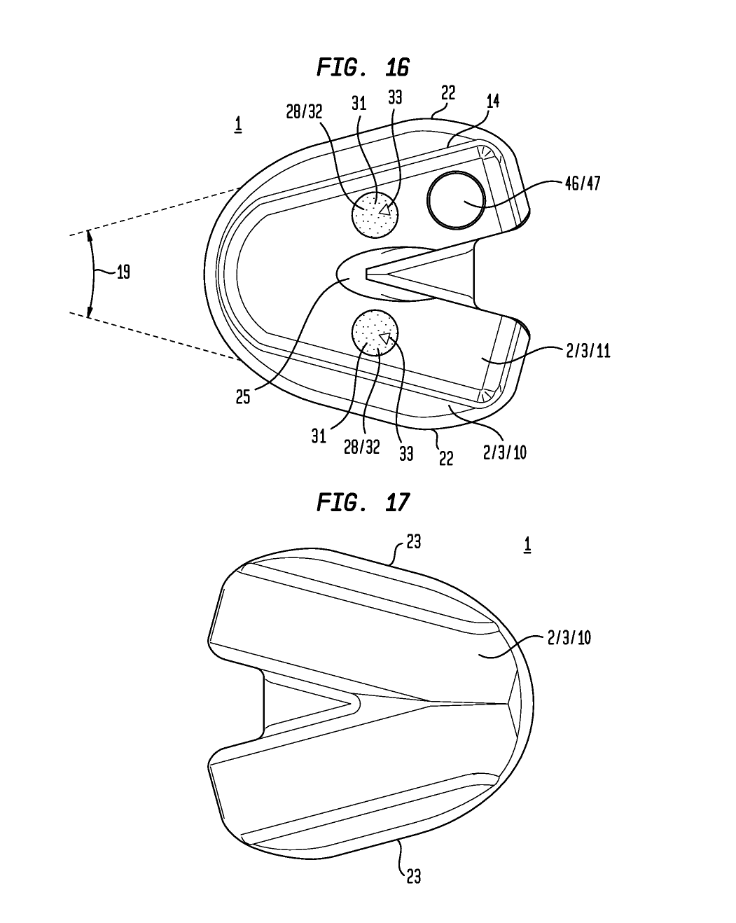

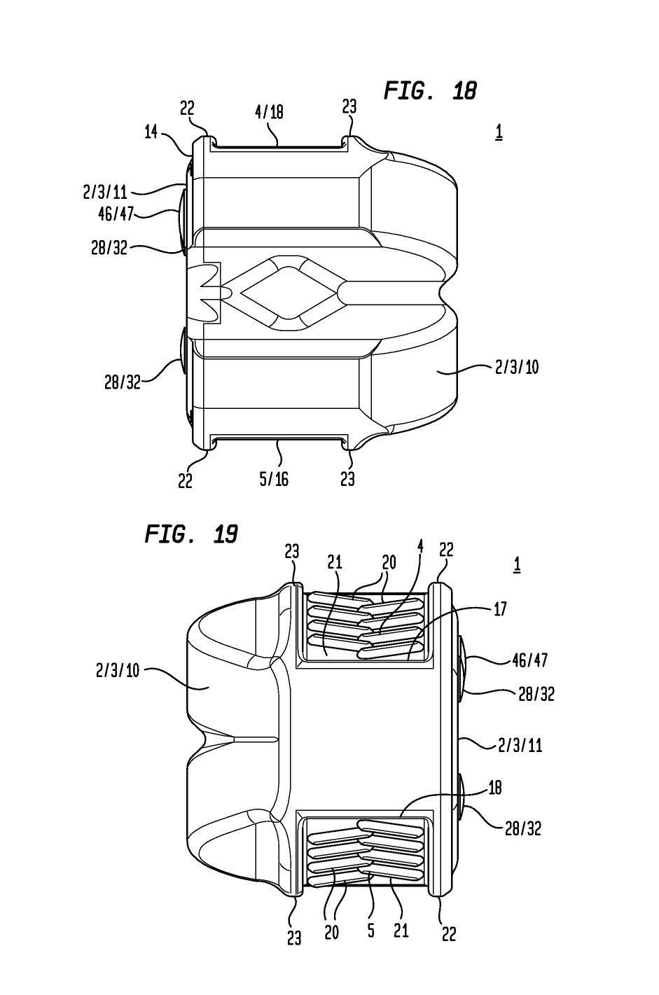

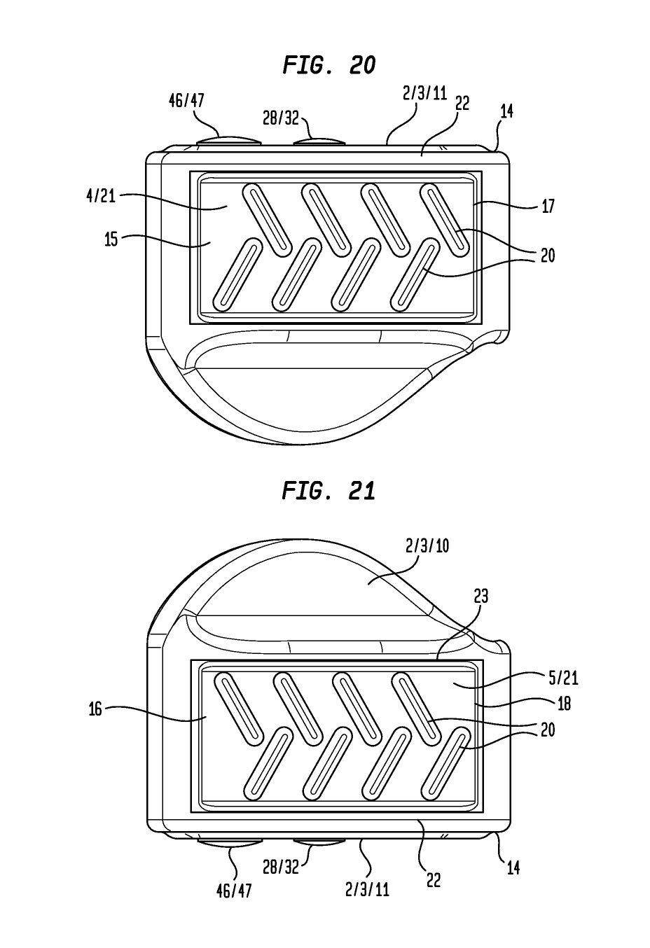

Now generally referring to FIGS. 1 through 25, embodiments of the inventive intraoral light illumination appliance (1) are shown which include an appliance body (2) having an external surface (3) configured to provide opposed bite surfaces (4)(5) to be engaged by the upper teeth (6) and he lower teeth (7) to prop open a mouth (8) of a patient (9), an internal surface (12) defining an interior chamber (13), and one or more translucent portions (28) which communicate between the internal surface (12) and the external surface (3) of the appliance body (2), and having disposed entirely inside the interior chamber (13) of the appliance body (2) a discrete power source (35) electrically connected to the light emitting element (34) operable to emit light (29) through the translucent portion (28) of the appliance body (2) to illuminate the mouth (8) of the patient (9).

The term “mouth” as used herein means the cavity lying at the upper end of the alimentary canal, bounded on the outside by the lips and inside by the oropharynx and containing the tongue, gums, and teeth.

The term “patient” as used herein broadly encompasses any animal which receives care utilizing an embodiment of the invention and in particular includes humans as shown in the illustrative example of FIG. 1.

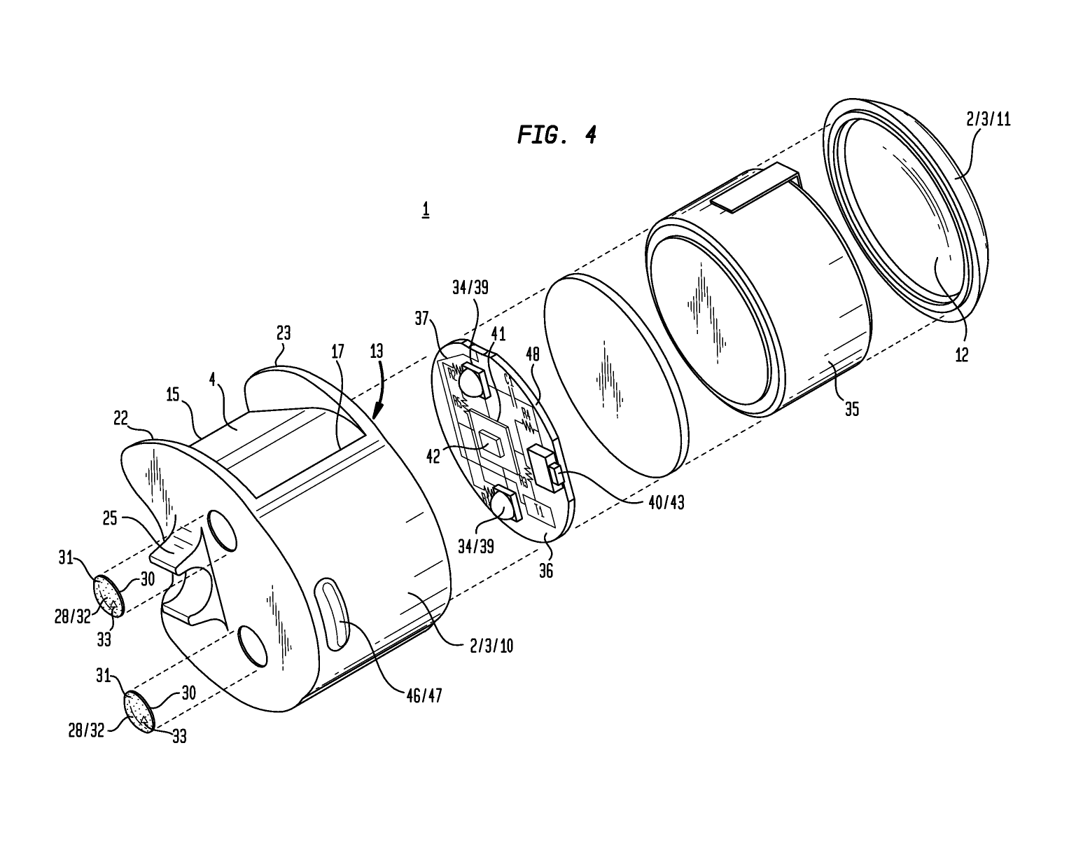

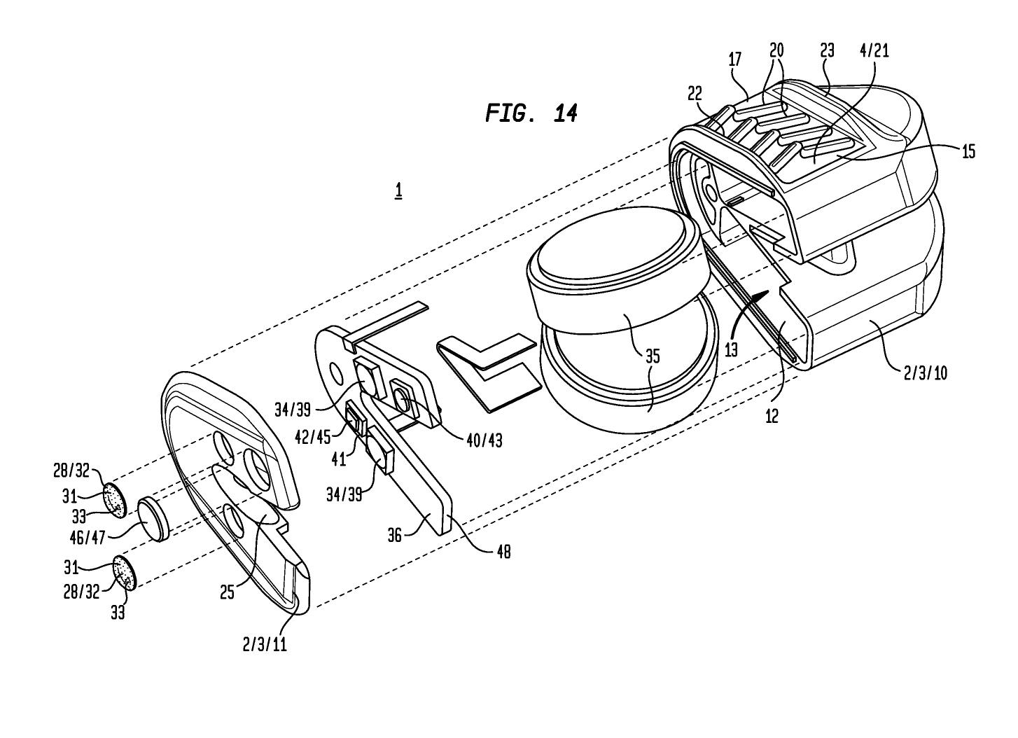

Now referring primarily to FIGS. 4 and 14, as to particular embodiments, the appliance body (2) can include a pair of body housings (10)(11) which sealably couple to provide the external surface (3) and the internal surface (12) defining the interior chamber (13) as a unitary appliance body (2). The unitary appliance body (2) can be can be separated at the juncture plane (14) to provide the pair of body housings (10)(11). The juncture plane (14) can located differently depending upon the configuration of the appliance body (2) and the method used to produce the appliance body (2).

The term “sealably couple” as used herein means that the pair of body housings (10)(11) join at a juncture plane (14) providing a seal substantially impermeable to solids and liquids, such as body fluids originating from within the patient (9) including as examples: blood, saliva, sweat, digestive fluids or the like; liquids originating external to the patient (9) such as water, disinfectants, biologic medications, dentifrices, pastes, fluorides, whiteners, or the like, and similar solids and liquids, whether individually or in various combinations, and as to particular embodiments substantially impermeable to solids and liquids even during sterilizing procedures such as autoclave procedures for destroying microorganisms. The unitary appliance body (2) can be can be separated at the juncture plane (14) to provide the pair of body housings (10)(11). The juncture plane (14) can located differently depending upon the configuration of the appliance body (2) and the method used to produce the appliance body (2).

The appliance body (2), or each of the pair of body housings (10)(11), can be fabricated, formed, molded, or otherwise produced from one or more of a wide variety of materials, such as: metal, plastic, glass, thermoplastic, thermoplastic composites, or the like, and as to particular embodiments, autoclavable materials, or combinations of materials, such as: polypropylene, fluorocarbon, polycarbonate, polymethylpentane, polyvinylchloride, polyethylene, polytetrafluoroethylene, silicone, polyacetal, elastomers, or mixtures or combinations thereof.

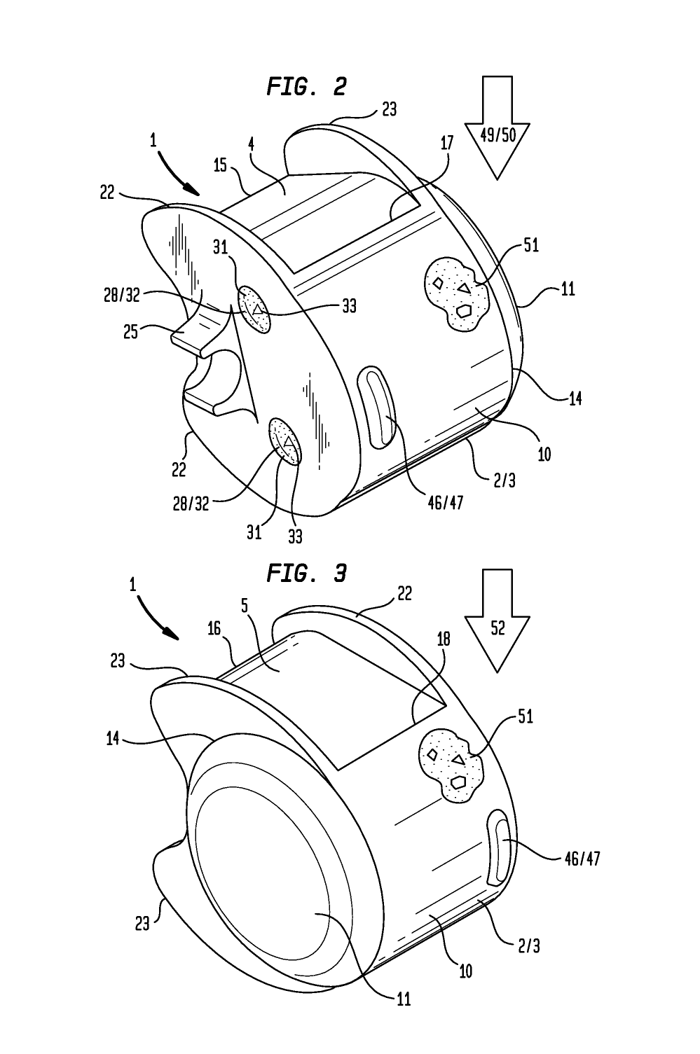

The term “autoclavable” as used herein means, with respect to any material, or combination of materials, or embodiments of the intraoral illumination appliance (1), retaining normal characteristics or operation of the material, combination of materials, or the intraoral illumination appliance (1) upon one or more exposures to pressures (50) and temperatures (49) within an autoclave sufficient to destroy microorganisms (51)(as illustrated in FIG. 2). Typically, autoclaves achieve temperatures (49) within range of about 100° C. and about 190° C. and pressures within the range of about 140 kPa and about 250 kPa depending upon whether a dry heat or a superheated steam is utilized in the autoclaving procedure which can be

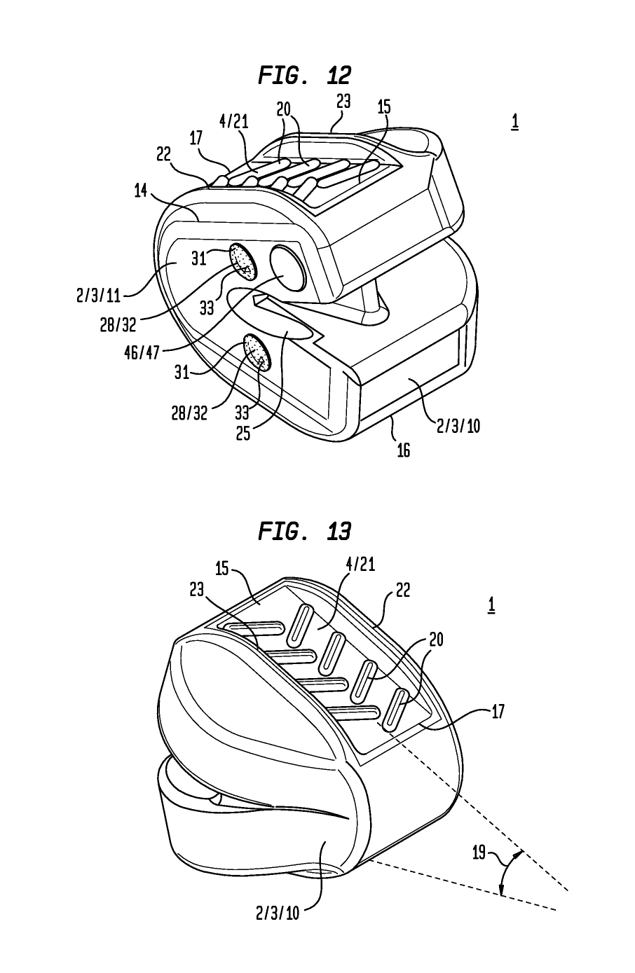

The external surface (2) of the inventive intraoral light illumination appliance (1) can have various configurations including embodiments which have a generally cylindrical configuration (as shown in the example of FIGS. 2 through 10) and embodiments which have a generally wedge configuration (as shown in the example of FIGS. 12 through 21); however, these illustrative examples are intended to provide a description sufficient for a person of ordinary skill in the art to produce a numerous and wide variety of appliance body (2) configurations which can be removably inserted in the mouth (8) and provide opposed bite surfaces (4)(5) which can be engaged by the upper teeth (6) and lower teeth (7) of a patient (9) to prop open the mouth (8).

Again referring primarily to FIGS. 2 through 10 and FIGS. 12 through 21, the opposed bite surfaces (4)(5) regardless of the general configuration of the appliance body (2) can be generally flat and disposed in angled relation to dispose the opposed anterior portions (15)(16) at a greater distance apart than the opposed posterior portions (17)(18) to provide a bite surface angle (19). While the bite surface angle (19) of the illustrative examples shown by the Figures, is between about 30 degrees and about 40 degrees; the examples are not intended to preclude embodiments of the invention which provide opposed bite surfaces (4)(5) in other configurations which correspondingly allow engagement by the upper teeth (6) and the lower teeth (7) of a patient (9).

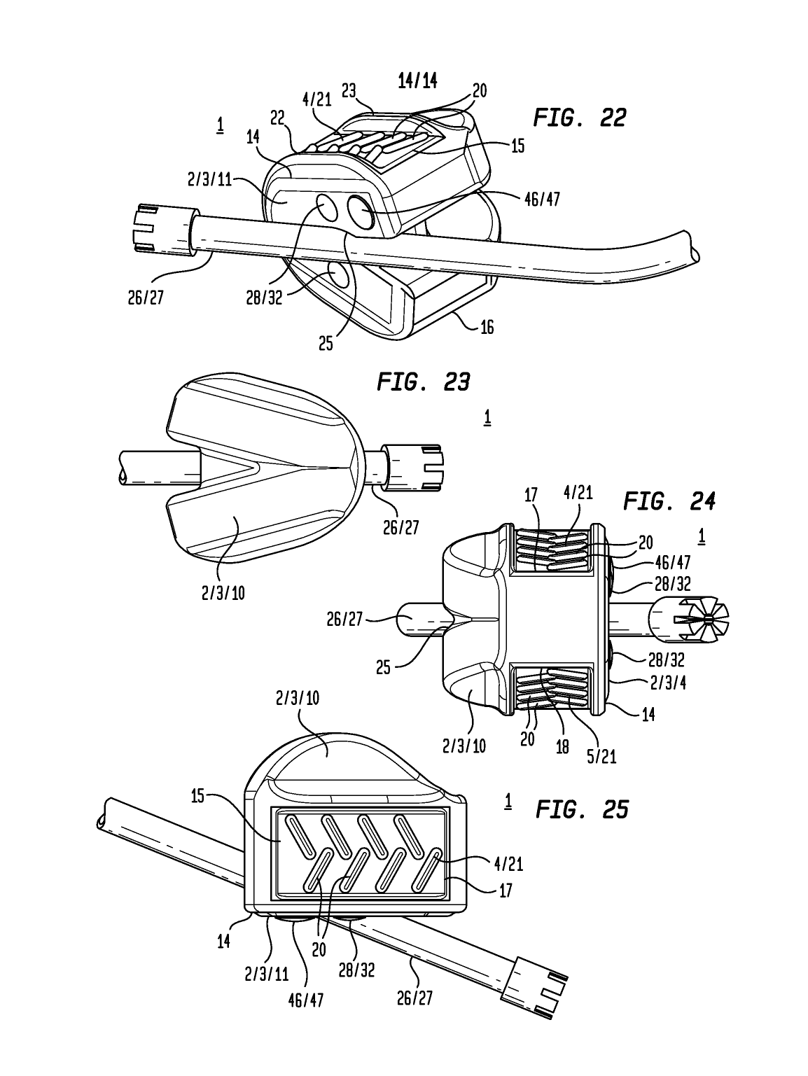

Now referring primarily to FIGS. 22, 24 and 25, embodiments of the opposed bite surfaces (4)(5) can further include patterned surface elements (20) which assist in maintaining the appliance body (2) at a fixed location between the upper teeth (6) and the lower teeth (7) during use. While the patterned surface elements (20) are shown in the figures as a herringbone pattern; the patterned surface elements (20) can be provided in any useful pattern of raised or recessed elements.

Additionally, particular embodiments can further include a bite pad (21) overlaying one or both of the opposed bite surfaces (4)(5). As to particular embodiments, the bite pad (21) can have a lesser hardness or durometer than the appliance body (2). As an illustrative example, the bite pad (21) can have a durometer of between about 60 D to about 90 D. The bite pad (21) can further include patterned surface elements (20). The bite pad (21) can be fabricated, formed, molded or otherwise produced from an elastomer such as polypropylene, polyisoprene, polybutadiene, chloroprene, butyl rubber, nitrile rubber, silicon rubber, or the like.

Again referring primarily to FIGS. 22, 24 and 25, particular embodiments of the appliance body (2) can further comprise one or a pair of flanges (22)(23) which extend from the appliance body (2) adjacent each of the opposed bite surfaces (4)(5) which during normal use of the intraoral light illumination appliance (1) positions the flange or a first of the pair of flanges (40(5) between the teeth (6)(7) and cheek (22) of the patient (9) or positions the flange or a second of the pair of flanges (23) between the teeth (6)(7) and the tongue (24) of the patient (9), respectively. The one or the pair of flanges (22)(23) serve to retain the appliance body (2) at a fixed location between the upper teeth (6) and the lower teeth (7) of the patient (9) during use.

Now referring primarily to FIGS. 1 through 22, particular embodiments of the appliance body (2) can further comprise a retention element (25) adapted to retain one or more discrete intraoral devices (26). While the Figures illustrate a retention element (25) adapted to retain a dental suction tube (27); this is not intended to limit configurations of the retention element (25) solely to retaining a suction tube (27) but rather provides sufficient description for a person of ordinary skill in the art to make and use configurations of the retention element (25) to retain a wide variety of oral surgery tools such as dental aspiration tubes, dental picks, or the like.

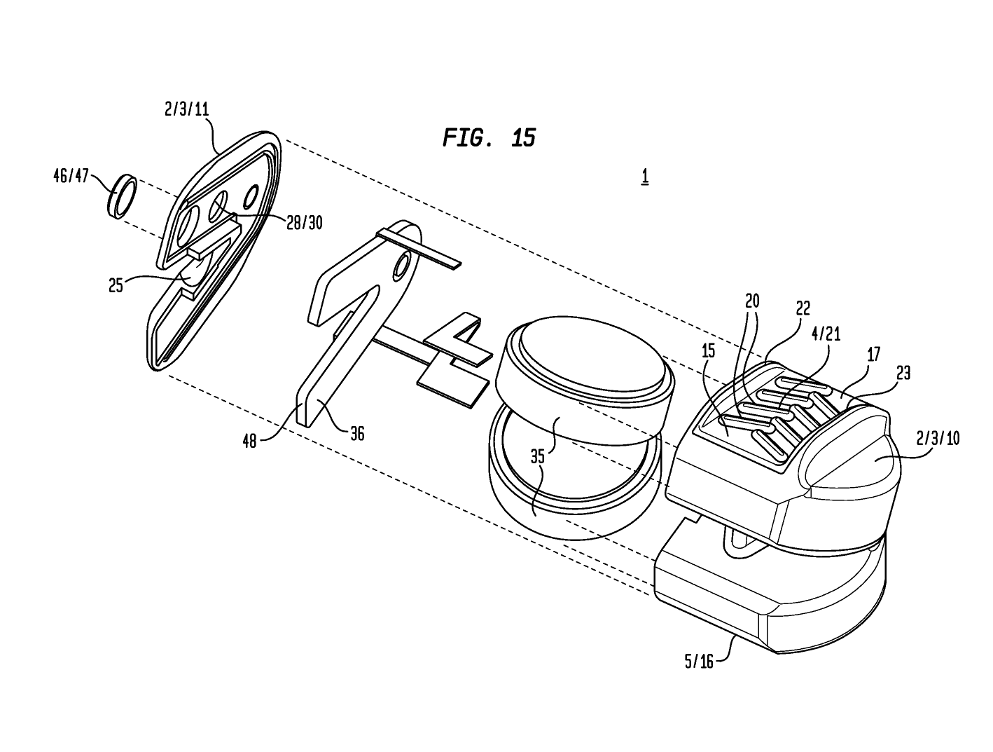

Now referring primarily to FIGS. 3, 4, 6, 12, 14, 15, and 16, embodiments of the appliance body (2) can further include one or more translucent portions (28). The term “translucent” as used herein means permitting light (29) to pass through. Translucent materials broadly encompass those materials which diffuse light as it passes through and those materials which transmit light so that objects or images can be seen as if there were no intervening material (also referred to as transparent materials). The appliance body (2), or each of the pair of body housings (10)(11), with one or more translucent portions (28) can be produced as a one-piece or the one or more translucent portions (28) can be produced as discrete light conductive elements sealably coupled to the appliance body (as shown in the examples of FIGS. 4 and 15).

The one or more translucent portions (28) of the appliance body (2), whether fabricated, formed, molded, or produced by other process(es), provides a configuration which can receive light (29) in the visible, ultraviolet, or infrared spectrum, separately or in combinations thereof, from one or more a light emitting elements (30). The translucent portion (28) can transmit the light (29) received at a translucent portion first face (30) internally, in whole or in part, as modified by any dopants (31) or colorants (33) included in the translucent material of the translucent portion (28), to be emitted at a translucent portion second face (32).

Various translucent materials can be utilized to produce the one or more translucent portions (28) of the appliance body (2), including, as illustrative examples: polystyrene, polycarbonate, polyvinylchloride, fluorocarbon, nylon, polystyrene, polyurethane, acrylic, polyethylene terphthalate, polyethersulfone, polymethylmethacrylate, polytetrafluoroethylene or the like; and as to particular embodiments, the translucent materials can be autclavable translucent materials such as: polypropylene, fluorocarbon, polycarbonate, polymethylpentane, polyvinylchloride, polyethylene, silicone, polyacetal, or mixtures or combinations thereof.

Particular embodiments of the translucent portion (28), as a consequence of the type of translucent material or the constructional form of the translucent portion (28)(or as a consequence of both), can achieve total internal reflection, substantial internal reflection, or the desired level of internal reflection of the light (29) incident on the translucent portion first face (30) which allows transmission of all, substantially all, or a desired amount of light (29) to the translucent portion second face (32) of the translucent portion (28).

The translucent materials can further include or contain one or more dopants (31). The term “dopant” as used herein means one or a plurality of similar or dissimilar trace impurity element(s) included separately or in various permutations and combinations in the translucent material at concentrations such that the amount of light (29) in portions of the spectrum received by the translucent portion first face (30) and transmitted to the translucent portion second face (32), whether in whole or in part, activates the one or more dopant(s)(31) which in turn fluoresce in a corresponding one or more wavelengths delivered to the translucent portion second face (32) as a color perceivable to the eye.

The translucent materials of the translucent portion (28) can further include or contain one or more colorants (33). The colorant can be combined in various permutations and combinations with the translucent material and one or more dopants (31) to achieve a desired color and fluorescence of the light (29) delivered at the translucent portion second face (32).

Now referring primarily to FIGS. 4, 5, 14 and 15, embodiments of the appliance body (2) can include an internal surface (12) defining an interior chamber (13). As to particular embodiments, a light emitting element (34) and a power source (35) electrically connected to the light emitting element (34) can be disposed entirely inside the interior chamber (13) of the appliance body (2) obviating the need for any external power source, power conduit, light pipe, or the like. Accordingly, these embodiments of the intraoral illumination appliance (1) can be used as shown in the example of FIG. 1 as a discrete unitary assembly independent of any external power source or light pipe; however, it is not intended that the illustrative examples shown in the Figures preclude other embodiments of the intraoral illumination appliance (1) which may further include a power source or light pipe external of the appliance body (2).

The light emitting element (34) can be located inside the interior chamber (13) to deliver all or a part of the light (29) emitted through the translucent portion (28) of the appliance body (2). As to certain embodiments, the light emitting element (34) can be mounted on an integrated circuit board (36) which supports and electrically interconnects the light emitting element (34) to the power source (35). The term “light emitting element” as used herein broadly encompasses one or more of any manner of light emitting element (34) which can be utilized to produce embodiments of the invention and illustrative examples includes: one or more of incandescent, halogen, grain-of-wheat, plasma, cold cathode, fluorescent, light emitting diodes, or organic light emitting diodes, or combinations thereof. As to particular embodiments, the light emitting element (34) can be a high power light-emitting diode which in a single package type can use electrical energy from about 500 milliwatts to about 500 watts. As an illustrative example, a suitable light emitting diode can be a cool white 5700K, 37 lumen, 120 degree dispersion angle with surface mount.

The term “power source” as used herein means any device that generates sufficient power, whether or not transformed, which can be electrically coupled to operate the light emitting element (34). As to particular embodiments, the power source (35) has a configuration that can be disposed entirely inside of the interior chamber (13) of the appliance body (2)(as shown in the examples of FIGS. 4 and 14). As an illustrative example, a suitable power source (35) can be a battery such as a coin type lithium cell, 3 volt, 1000 mAh capacity with two placed in series to provide 6 volts. As to other embodiments of the invention having a power source (35) external to the intraoral illumination appliance (1), a suitable power source (35) can be conventional 110 volt alternating current (AC) converted to 6 volt or to 24 volt direct current (DC) by use of an AC to DC transformer.

Now referring primarily to FIGS. 4 and 14, an exemplary circuit (37) can include a power source (35)(whether as one or more power sources whether connected in series or in parall) delivering a direct current, and two components connected in series: a current-limiting resistor (38) and a light emitting diode (39). Although simple, this circuit (37) is not necessarily the most energy efficient circuit to drive a light emitting diode (39), since energy can be lost in the resistor (38), and more complicated circuits can be used to improve energy efficiency and are encompassed by the invention.

Again referring primarily to FIGS. 4 and 14, as to particular embodiments, a switch (40) can be electrically coupled between the power source (35) and the light emitting element (34) to open and close the circuit (37). As to particular embodiments, the circuit (37), switch (40) and light emitting element (34) can be mounted on the integrated circuit board (36). The integrated circuit board periphery (48) can be configured to maintain the integrated circuit board (36) at a fixed location in relation to the internal surface (12) of the appliance body (2) and the one or more light emitting elements (34) and the switch (40) can be mounted at fixed locations on the integrated circuit board (36) to locate the light emitting elements (34) to deliver emitted light (29) to the translucent portion first face (30), and the locate the switch (40) proximate the internal surface (12) of the appliance body (2). The switch (40) can have a plurality of switch conditions (43) selectable to correspondingly select one or more of a plurality of light emitting conditions (44) of one or more light emitting elements (34) such as an on condition (44a), an off condition (44b), a light wavelength frequency condition (44c), and a light wavelength amplitude condition (44d) or combinations thereof. The term “off condition” as used herein means an open circuit (37) between the power source (35) and the light emitting element (34). The term “on condition” means a closed circuit (37) between the power source and the light emitting element (34). The term “light wavelength frequency condition” as used herein means one or more light wavelength frequencies emitted by a light emitting element (34) selected from within a range of wavelength frequencies by operation the switch (40). For example, operation of the switch (40) can be utilized to alter the light wavelength frequency to correspondingly alter the color of the light (29) emitted by the light emitting element (34). The term “light wavelength amplitude” as used herein means one or more light wavelength amplitudes emitted by a light emitting element (34) selected from within a range of wavelength amplitudes by operation the switch (40). For example, operation of the switch (40) can be utilized to alter the light wavelength amplitude to correspondingly alter brightness of the light (29) emitted by the light emitting element (34).

Again referring primarily to FIGS. 4 and 14, particular embodiments can further include a logic element (41) containing a logic function (42) interactive with operation of the switch (40). As to particular embodiments, the logic function (42) can be interactive with the operation of the switch (40) to select between various light emitting element conditions (43) for each one of the light emitting elements (34) such as an on condition (44a), an off condition (44b), a light wavelength frequency condition (44c), and a light wavelength amplitude condition (44d) or combinations thereof, as above described. The logic element (41) can be mounted on the integrated circuit board (36).

Now referring primarily to FIGS. 2 through 8, embodiments can further include a movable portion (46) of the appliance body (2) which, upon sufficient forcible urging, moves inward to operably contact the switch (40) located within the interior chamber (13). As to particular embodiments, the movable portion (46) can be a depressible or deformable member (47) sealably coupled to the appliance body (2). The deformable member (47) deforms sufficiently toward the interior chamber (13) to operably contact the switch (40). As to certain embodiments, the movable portion (46) or deformable member (47) can be one-piece with the housing body (2) a portion of which can have a thinness which upon sufficient forcible urging moves or deforms to contact the switch (40). The movable portion (46) or deformable member (47) can be produced from an autoclavable material, as above described.

Now referring primarily to FIG. 1, an illustrative method of using a particular embodiment of the intraoral illumination appliance (1) which includes inserting the appliance (1) into a mouth (8) of a patient (9). The appliance (1) including, as above described, an appliance body (2) having an external surface (3) including opposed bite surfaces (4)(5); an internal surface (12) defining an interior chamber (13); and at least one translucent portion (28) which communicates between the internal surface (12) and the external surface (3) of the appliance body (2); at least one light emitting element (34); and a power source (35) electrically connected to the light emitting element (34), the light emitting element (34) and the power source (35) disposed entirely inside the interior chamber (13) of the appliance body (20). The patient (9) by engaging upper and lower teeth (6)(7) with the opposed bite surfaces (4)(5) of the appliance body (2) props open the mouth (8). Emitting light (29) through the at least one translucent portion (28) of the appliance (1) illuminates the mouth (8) of the patient (9).

As to particular embodiments, the method can further include operating a switch (40) electrically couples the power source (35) to light emitting element (34). By operating the switch (40) between a plurality of switch conditions (43) a correspondingly plurality of light emitting conditions (44) of at least one light emitting element (34) can be selected comprising or selected from the group consisting of: an on condition (44a), an off condition (44b), a light wavelength frequency condition (44c), and a light wavelength amplitude condition (44d) or combinations thereof, as above described.

As to particular embodiments, the method can further include retaining one or more discrete intraoral devices (26) in a retention element (25) coupled to the external surface (3) of the appliance (1).

As to particular embodiments, the method can further include exposing the appliance to temperature (49) or pressure (50) sufficient to destroy microorganisms, the appliance (1) retaining normal operation upon one or more exposures to the temperature or pressure sufficient to destroy microorganisms (51)(as shown in the example of FIG. 2). As to certain embodiments of the method the temperature (49) or the pressure (50) sufficient to destroy microorganisms (51) comprises autoclave conditions sufficient to destroy microorganisms (51), as above described.

As to particular embodiments, the method can further include exposing the appliance (1) to one or more fluids (52) capable of destroying microorganisms (51), the appliance (1) retaining normal operation upon one more exposures to the one or more fluids (52) capable of destroying said microorgaisms (51). The one or more fluids can comprise or be selected from the group consisting of: ethanol, isopropylalcohol, formaldehyde, gluteraldehyde, phenol, o-phenylphenol, hexochlorophene, amylmetacreasol, 2.4 dichlorobenzyl alcohol, silver ion, copper ion, sodium hypochlorite, chloramine, chloromine T, chlorine dioxide, hydrogen peroxide, ozone, peracetic acid, performic acid, potassium permanganate, potassium peroxymonosulfate, or the like, or combinations thereof.

As can be easily understood from the foregoing, the basic concepts of the present invention may be embodied in a variety of ways. The invention involves numerous and varied embodiments of an intraoral light emitting appliance and methods for making and using such intraoral light emitting appliance including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “light emitter” should be understood to encompass disclosure of the act of “emitting light”—whether explicitly discussed or not—and, conversely, were there effectively disclosure of the act of “emitting light”, such a disclosure should be understood to encompass disclosure of a “light emitter” and even a “means for emitting light.” Such alternative terms for each element or step are to be understood to be explicitly included in the description.

In addition, as to each term used it should be understood that unless its utilization in this application is inconsistent with such interpretation, common dictionary definitions should be understood to included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the intraoral light emitting appliances herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. An intraoral illumination appliance, comprising:
an appliance body including:
an external surface having opposed bite surfaces adapted to be engaged by upper and lower teeth to prop open a mouth of a patient;
an internal surface defining an interior chamber; and
a translucent portion which communicates between said internal surface and said external surface of said appliance body;
at least one light emitting element; and
a power source electrically connected to said light emitting element, said light emitting element and said power source disposed entirely inside said interior chamber of said appliance body, said light emitting element having a location inside said interior chamber to emit light through said translucent portion of said appliance body.

2. The intraoral illumination appliance of claim 1, wherein said translucent portion comprises one or more discrete light conductive elements sealably coupled to said appliance body.

3. The intraoral illumination appliance of claim 2, wherein said one or more light conductive elements further include one or more dopants.

4. The intraoral illumination appliance of claim 2, wherein said one or more light conductive elements further include one or more colorants.

5. The intraoral illumination appliance of claim 1, further comprising one or more flanges which extend from said appliance body adjacent each of said opposed bite surfaces.

6. The intraoral illumination appliance of claim 5, further comprising a bite pad overlaying each of said bite surfaces, said bite pad having a lesser durometer than said appliance body.

7. The intraoral illumination appliance of claim 6, further comprising one or more patterned surface elements coupled to said bite pad.

8. The intraoral illumination appliance of claim 1, further comprising a switch disposed inside said interior chamber, said switch interruptably electrically coupling said power source to said light emitting element.

9. The intraoral illumination appliance of claim 8, further comprising a movable portion of said appliance body which upon forcible urging moves sufficiently toward said interior chamber to operably contact said switch.

10. The intraoral illumination appliance of claim 9, wherein said movable portion of said appliance body comprises a deformable member sealably coupled to said appliance body which upon forcible urging sufficiently deforms toward said interior chamber to operably contact said switch.

11. The intraoral illumination appliance of claim 8, wherein said switch has a plurality of switch conditions selectable to correspondingly select one or more of a plurality of light emitting conditions of said at least one light emitting element.

12. The intraoral illumination appliance of claim 11, wherein said one or more of said plurality of light emitting conditions is selected from the group consisting of: an on condition, an off condition, a light wavelength frequency condition, and a light wavelength amplitude condition.

13. The intraoral illumination appliance of claim 12, further comprising a logic element containing a logic function interactive with operation of said switch.

14. The intraoral illumination appliance of claim 13, wherein said logic function comprises a light emitting element selection function which allows selection of one or more of said plurality of light emitting conditions.

15. The intraoral illumination appliance of claim 14, further comprising an integrated circuit board which supports and electrically interconnects said light emitter, said switch and said logic element.

16. The intraoral illumination appliance of claim 1, wherein said light emitting element comprises a light emitting diode.

17. The intraoral illumination appliance of claim 16, wherein said power source comprises at least one battery.

18. The intraoral illumination appliance of claim 1, further comprising a retention element adapted to retain one or more discrete intraoral devices.

19. The intraoral illumination appliance of claim 1, wherein said appliance body comprises a pair of body housings which sealably couple to provide said external surface and said an internal surface defining said interior chamber.

20. The intraoral illumination appliance of claim 1, wherein said intraoral illumination appliance retains normal operation upon one more exposures to temperature or pressure sufficient to destroy microorganisms.

21. The intraoral illumination appliance of claim 20, wherein said temperature or said pressure which destroys microorganisms comprises autoclave conditions which destroy microorganisms.

22. The intraoral illumination appliance of claim 21, wherein said temperature has a range of about 100° C. and about 190° C. and wherein said pressure has a range of about 140 kPa and about 250 kPa.

23. The intraoral illumination appliance of claim 1, wherein said intraoral illumination appliance retains normal operation upon one more exposures to one or more fluids capable of destroying microorganisms.

24. The intraoral illumination appliance of claim 23, further comprising selecting said one or more fluids from the group consisting of: ethanol, isopropylalcohol, formaldehyde, gluteraldehyde, phenol, o-phenylphenol, hexochlorophene, amylmetacreasol, 2.4 dichlorobenzyl alcohol, silver ion, copper ion, sodium hypochlorite, chloramine, chloromine T, chlorine dioxide, hydrogen peroxide, ozone, peracetic acid, performic acid, potassium permanganate, and potassium peroxymonosulfate.

25-57. (canceled)
